(19) United States
(12) Patent Application Publication
(10) Pub. No.: US 2019/0104946 A1
(43) Pub. Date: Apr. 11, 2019

(54) PRESSURE SENSOR WITH INTEGRATED LEVEL REFERENCE

(71) Applicant: Edwards Lifesciences Corporation, Irvine, CA (US)

(72) Inventor: Blake W. Axelrod, Sierra Madre, CA (US)

(21) Appl. No.: 16/140,130

(22) Filed: Sep. 24, 2018

Related U.S. Application Data
(60) Provisional application No. 62/571,120, filed on Oct. 11, 2017.

(51) Int. Cl. A61B 5/023; G01L 1/18; A61B 5/021; A61B 5/0225

(52) U.S. Cl. CPC A61B 5/023; A61B 5/0225; A61B 5/02141; G01L 1/18

(57) ABSTRACT

Disclosed is a blood pressure measurement device for a patient at a patient measurement site, comprising: a housing; and a pressure sensing chip mounted in the housing that is attachable to the patient measurement site. The pressure sensing chip may include a pressure transducing member. The pressure sensing chip may be configured to measure the patient's blood pressure based upon: 1) pressure applied by the patient's blood against the pressure transducing member at a first side of the pressure transducing member; and 2) gravity generated pressures over a height difference between the patient's heart level and a point of blood pressure measurement applied against the pressure transducing member at a second side of the pressure transducing member.

### CROSS-REFERENCE TO RELATED APPLICATIONS

This application claims priority to U.S. Provisional Application Ser. No. 62/571,120, filed Oct. 11, 2017, the contents of which are incorporated herein by reference in its entirety.

### BACKGROUND

### Field

Embodiments of the invention relate to a method, apparatus, and system for measuring blood pressure.

### Relevant Background

There are presently many different types of pressure sensor configurations for measuring blood pressure and blood pressure waveforms of a patient.

As one example, a Disposable Pressure Transducer (DPT) may be used with arterial and other catheters. It is a low fidelity, low cost, disposable pressure sensor. The DPT housing mounts on an IV pole and connects to the catheter through long tubing. The housing is a flow through device that keeps the pressure sensor patent by maintaining a constant pressure upstream of the sensor. Additionally, fluid can be added or withdrawn from the patient through the sensor. The DPT is a differential pressure sensor that measures relative to the atmospheric pressure in the room. To compensate for pressures generated by height differences (gravity) between the catheter and the patient's heart, the DPT is positioned on the IV pole at the patient's heart level.

As another example, a finger cuff pressure sensor may be used to measure the pressure generated with an air system in a volume clamp cuff. This is a common air pressure sensor that measures the air pressure in the volume clamp cuff relative to atmospheric pressure in the room. The sensor may be located within a wrist unit.

A second pressure sensor, a Heart Reference Sensor (HRS), may be utilized with the finger cuff system to compensate for pressures generated by height differences between the patient's finger and heart. The HRS connects an oil filled bladder located at the patient's heart level to a pressure sensor located at the patient's finger or wrist unit through an oil filled tube. The gravity generated pressures between the patient's heart level and finger level are measured by the HRS and subtracted from the cuff pressure sensor in the system's data processing software/algorithms

The DPT's strengths are its low cost and its high modularity—it can easily be connected to a wide variety of catheters through the long tubing and a luer fitting. The two primary shortcomings of the DPT are the data losses due to the tubing and the process of leveling the DPT with the patient's heart on the IV pole. The long tubing introduces noise and artifacts due to mechanical resonances. To remove these effects, the sensor's data may be filtered, but this also removes significant higher frequency information from the data signal. Blood pressure waveforms are often processed in real-time with algorithms that calculate hemodynamic and physiological parameters such as Stroke Volume Variation and Cardiac Output. The loss of information slows algorithm convergence, and leaves the algorithm unable to track patients with arrhythmias and other effects. Further, the heart level system adds work to the clinician's workflow and doesn't track the patient's movements.

On the other hand, the finger cuff system uses two pressure sensors and combines the results in order to measure blood pressure that is compensated for by the patient's heart level and atmospheric pressure. Using two sensors is expensive and complicates manufacturing.

Therefore, there is a need for improved blood pressure measurement devices.

### SUMMARY

Embodiments of the invention may relate to a blood pressure measurement device for a patient at a patient measurement site, comprising: a housing; and a pressure sensing chip mounted in the housing that is attachable to the patient measurement site. The pressure sensing chip may include a pressure transducing member. The pressure sensing chip may be configured to measure the patient's blood pressure based upon: 1) pressure applied by the patient's blood against the pressure transducing member at a first side of the pressure transducing member; and 2) gravity generated pressures over a height difference between the patient's heart level and a point of blood pressure measurement applied against the pressure transducing member at a second side of the pressure transducing member.

### BRIEF DESCRIPTION OF THE DRAWINGS

FIG. 1 is a block diagram illustrating an example blood pressure measurement device, according to one embodiment of the invention.

FIG. 2 is a cross-section view of an example blood pressure measurement device, according to one embodiment of the invention.

FIG. 3 is a diagram illustrating an example blood pressure measurement system in which embodiments of the invention may be utilized.

FIG. 4 is a flowchart illustrating an example method for measuring a blood pressure utilizing a single blood pressure measurement device, according to one embodiment of the invention.

### DETAILED DESCRIPTION

Embodiments of the invention may relate to a blood pressure measurement device for a patient at a patient measurement site, comprising: a housing; and a pressure sensing chip mounted in the housing that is attachable to the patient measurement site. The pressure sensing chip may include a pressure transducing member. The pressure sensing chip may be configured to measure the patient's blood pressure based upon 1) pressure applied by the patient's blood against the pressure transducing member at a first side of the pressure transducing member and 2) gravity generated pressures over a height difference between the patient's heart level and a point of blood pressure measurement applied against the pressure transducing member at a second side of the pressure transducing member.

The pressure transducing member may include a membrane that includes a piezo resistive strain sensor such that a patient's blood abutting against the membrane results in a deformation of the membrane which is measured as a change in resistance in the piezo resistive strain sensor and is measured by the pressure sensing chip for measuring the patient's blood pressure. Any liquid with the same density as blood, typically around 1060 kg/m³, may be used as a measuring liquid abutting against the second side of the pressure transducing member to compensate for gravity generated pressures over a height difference between the patient's heart level and the point of blood pressure measurement. While any liquid with the same density as blood will correctly transfer gravity generated pressures to the pressure transducing member, it is generally preferred that the liquid be inert and biocompatible. It should be appreciated that oil or any suitable liquid may be utilized.

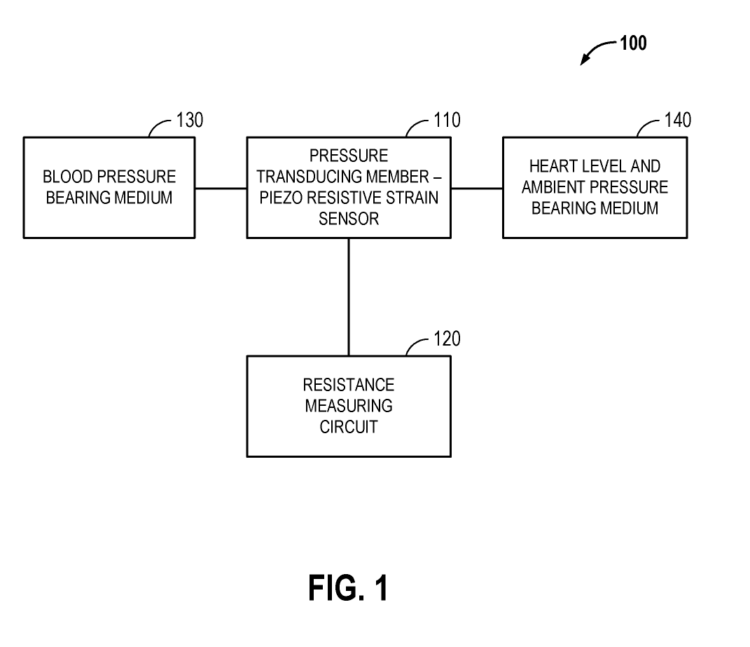

Referring to FIG. 1, a block diagram illustrating an example blood pressure measurement device 100, according to one embodiment of the invention, is shown. The blood pressure measurement device 100 may comprise a pressure transducing member 110 that may include a piezo resistive strain sensor. The pressure transducing member may be a deformable membrane. A blood pressure bearing medium 130 may be allowed access to a first side of the membrane, and a heart level and ambient pressure bearing medium 140 may be allowed access to a second side of the membrane opposite the first side. Thus, the membrane may deform under the joint influence of the blood pressure bearing medium 130 and the heart level and ambient pressure bearing medium 140. In other words, the effect on the membrane caused by the gravity generated pressure over a height difference between the heart level and the membrane height in the blood pressure bearing medium may be offset by the effect on the membrane caused by the heart level and ambient pressure bearing medium. As a result, the degree of membrane deformation is a function of the patient's blood pressure alone, and is independent of the gravity generated pressure.

The resistance in the piezo resistive strain sensor 110 is a function of membrane deformation. Thus, the patient's blood pressure may be measured indirectly through the measurement of the resistance in the piezo resistive strain sensor 110. A resistance measuring circuit 120 may be utilized to measure the resistance in the piezo resistive strain sensor. In one embodiment, the resistance measuring circuit 120 may comprise a Wheatstone bridge circuit. The output signal of the resistance measuring circuit 120 may be fed into a pressure sensing and data processing monitor that processes the output signal, determines the patient's blood pressure, and displays the patient's blood pressure to clinicians.

In one embodiment, pressure transducing member—piezo resistive strain sensor 110 and the resistance measuring circuit 120 may be incorporated into a silicon pressure sensing chip.

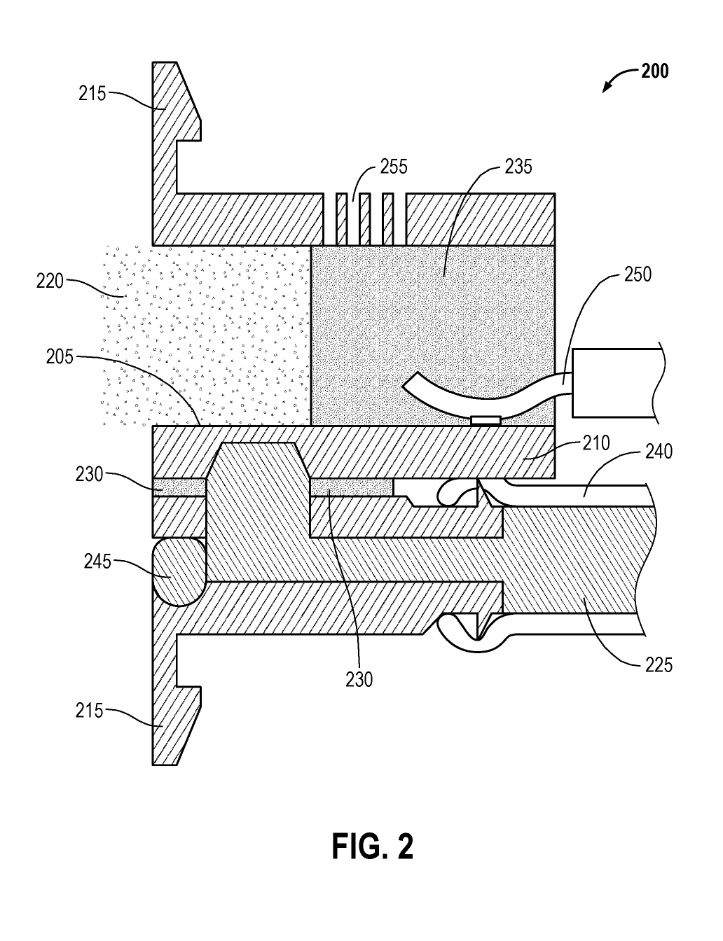

Referring to FIG. 2, a cross-section view of an example blood pressure measurement device 200, according to one embodiment of the invention, is shown. The blood pressure measurement device 200 may comprise a deformable membrane 205. Piezo resistive strain sensor(s) may be utilized to measure the membrane deflection. The piezo resistive effect is a change in the electrical resistivity of a semiconductor or metal when mechanical strain is applied. In one embodiment, the resistance in the piezo resistive sensors, which changes as a function of the membrane deflection, may be measured utilizing a Wheatstone bridge circuit. Therefore, a pressure difference across the membrane 205 results in a deformation of the membrane 205 that can be measured based upon a change in resistance in the piezo resistive strain sensor(s) by the silicon pressure sensing chip 210.

In particular, the pressure sensing chip 210 includes the pressure transducing membrane 205 (e.g., the deformable membrane 205 utilizing piezo resistive strain sensors) and the pressure sensing chip 210 measures the membrane deflection. The pressure sensing chip 210 may be packaged into a plastic housing 215 that allows the blood pressure bearing media 220—e.g., blood or air—access to a first side of the pressure transducing membrane 205 and the heart level and ambient pressure bearing media 225 access to a second side (opposite the first side) of the membrane 205.

In one embodiment, the housing 215 may be made of two pieces that are attached together and sealed with silicone gaskets 230 around the pressure sensing chip 210. The blood pressure side (e.g., the first side) may include a silicone plug or seal 235 (e.g., silicone gasket, air vent, and wire strain relief) that allows air to escape from the pressure sensing region through perforations 255 in the housing once the pressure measurement device 200 is attached to a catheter and exposed to the patient's blood pressure. Thus, in one embodiment, the blood pressure measurement device 200 may be attached to a catheter or another suitable measurement site. The heart level side (e.g., the second side) may include a connection for a liquid filled tube 240 and a sealing port 245 (e.g., a silicone or viton plug) to close the liquid filled tube. As has been described, it should be appreciated that oil or any suitable liquid may be utilized. Electrical connections may be made directly to the pressure sensing chip 210 via a wire 250. The wire 250 may be connected to the pressure sensing chip 210 at a connector outside the pressure sensing region and may enable direct electrical connections from the pressure sensing chip 210 to a pressure sensing and data processing monitor (e.g., via a cable).

Therefore, the pressure sensing chip 210 may be configured to measure the patient's blood pressure based upon: 1) pressure applied by the patient's blood against the membrane 205 at a first side of the membrane 205 and 2) gravity generated pressures over a height difference between the patient's heart level and a point of blood pressure measurement applied against the membrane 205 at a second side of the membrane 205.

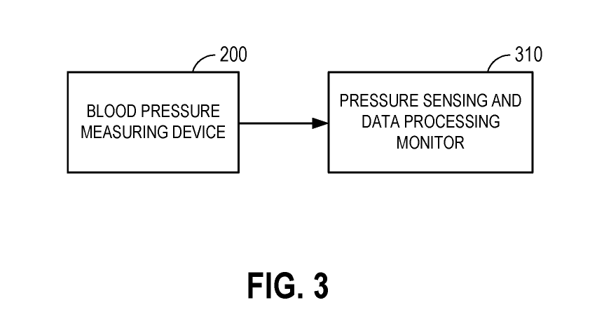

Referring to FIG. 3, a diagram illustrating an example blood pressure measurement system 300 in which embodiments of the invention may be utilized, is shown. The blood pressure measurement system 300 comprises the blood pressure measurement device 200 and a pressure sensing and data processing monitor 310. The pressure sensing and data processing monitor 310 may comprise appropriate hardware or an appropriate combination of hardware and software that enables it to receive signals outputted by the blood pressure measurement device 200, determines the patient's blood pressure based on the signals received from the blood pressure measurement device 200, and displays the patient's blood pressure to clinicians.

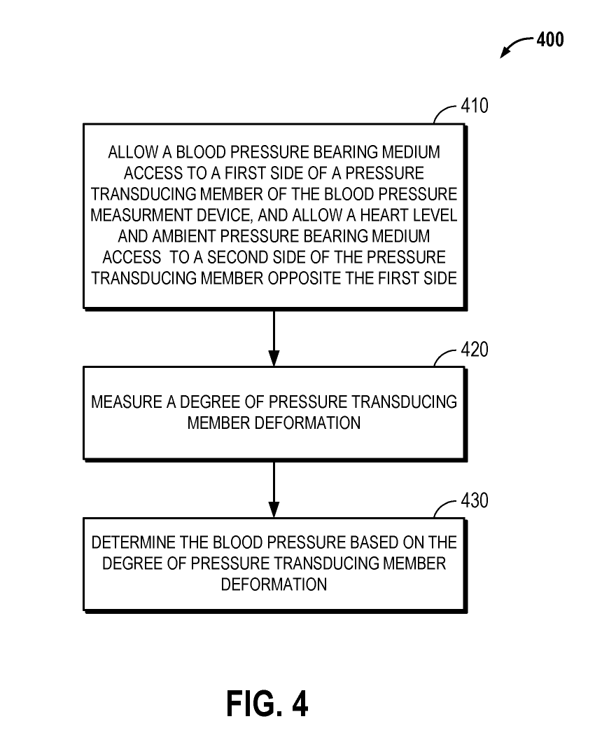

Referring to FIG. 4, a flowchart illustrating an example method 400 for measuring a blood pressure, utilizing a single blood pressure measurement device, according to one embodiment of the invention, is shown. At block 410, a blood pressure bearing medium may be allowed access to a first side of a pressure transducing member of the blood pressure measurement device, and a heart level and ambient pressure bearing medium may be allowed access to a second side of the pressure transducing member opposite the first side. At block 420, a degree of pressure transducing member deformation may be measured. The degree of pressure transducing member deformation may be measured electrically with a piezo resistive strain sensor. At block 430, the blood pressure may be determined based on the degree of pressure transducing member deformation.

Therefore, embodiments of the invention eliminate the need for long tubing that degrades the DPT pressure signal in DPT systems. This enables faster algorithm convergence and other high resolution data benefits. Further, it simplifies the operating room (OR) environment by eliminating cables and simplifying clinician setup. Further, embodiments of the invention reduce cost associated with finger cuff systems by reducing the number of required pressure sensors from two to one.

It should be appreciated that aspects of the invention previously described may be implemented in conjunction with the execution of instructions by processors, circuitry, controllers, control circuitry, etc. As an example, control circuity may operate under the control of a program, algorithm, routine, or the execution of instructions to execute methods or processes (e.g., method 400 of FIG. 4) in accordance with embodiments of the invention previously described. For example, such a program may be implemented in firmware or software (e.g. stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A blood pressure measurement device for a patient attached at a patient measurement site, comprising:
a housing; and
a pressure sensing chip mounted in the housing that is attachable to the patient measurement site, the pressure sensing chip including a pressure transducing member, the pressure sensing chip configured to measure the patient's blood pressure based upon pressure applied by the patient's blood against the pressure transducing member at a first side of the pressure transducing member and gravity generated pressures over a height difference between the patient's heart level and a point of blood pressure measurement applied against the pressure transducing member at a second side of the pressure transducing member.

2. The blood pressure measurement device of claim 1, wherein the pressure transducing member includes a membrane that includes a piezo resistive strain sensor such that a patient's blood abutting against the membrane results in a deformation of the membrane which is measured as a change in resistance in the piezo resistive strain sensor and is measured by the pressure sensing chip for measuring the patient's blood pressure.

3. The blood pressure measurement device of claim 2, wherein the change in resistance in the piezo resistive strain sensor is measured by the pressure sensing chip with a Wheatstone bridge circuit.

4. The blood pressure measurement device of claim 1, wherein oil is used as a measuring liquid abutting against the second side of the pressure transducing member to compensate for gravity generated pressures over a height difference between the patient's heart level and the point of blood pressure measurement.

5. The blood pressure measurement device of claim 1, wherein a liquid with density matched to blood pressure is used as a measuring liquid abutting against the second side of the pressure transducing member to compensate for gravity generated pressures over a height difference between the patient's heart level and the point of blood pressure measurement.

6. The blood pressure measurement device of claim 1, further comprising a silicone plug or seal located between the pressure sensing chip and the housing to allow air to escape from a region surrounding the pressure sensing chip when attached to a catheter to measure the patient's blood pressure.

7. The blood pressure measurement device of claim 1, further comprising a wire connectable to the pressure sensing chip for direct electrical connections from a connector of the pressure sensing chip outside a pressure sensing region to a pressure sensing and data processing monitor outside of a pressure sensing region.

8. A blood pressure measurement system comprising:
a blood pressure measurement device attached at a patient measurement site of a patient, the blood pressure measurement device, comprising:
a housing; and
a pressure sensing chip mounted in the housing that is attachable to the patient measurement site, the pressure sensing chip including a pressure transducing member, the pressure sensing chip configured to measure the patient's blood pressure based upon pressure applied by the patient's blood against the pressure transducing member at a first side of the pressure transducing member and gravity generated pressures over a height difference between the patient's heart level and a point of blood pressure measurement applied against the pressure transducing member at a second side of the pressure transducing member.

9. The blood pressure measurement system of claim 8, wherein the pressure transducing member includes a membrane that includes a piezo resistive strain sensor such that a patient's blood abutting against the membrane results in a deformation of the membrane which is measured as a change in resistance in the piezo resistive strain sensor and is measured by the pressure sensing chip for measuring the patient's blood pressure.

10. The blood pressure measurement system of claim 9, wherein the change in resistance in the piezo resistive strain sensor is measured by the pressure sensing chip with a Wheatstone bridge circuit.

11. The blood pressure measurement system of claim 8, wherein oil is used as a measuring liquid abutting against the second side of the pressure transducing member to compensate for gravity generated pressures over a height difference between the patient's heart level and the point of blood pressure measurement.

12. The blood pressure measurement system of claim 8, wherein a liquid with density matched to blood pressure is used as a measuring liquid abutting against the second side of the pressure transducing member to compensate for gravity generated pressures over a height difference between the patient's heart level and the point of blood pressure measurement.

13. The blood pressure measurement system of claim 8, further comprising a silicone plug or seal located between the pressure sensing chip and the housing to allow air to escape from a region surrounding the pressure sensing chip when attached to a catheter to measure the patient's blood pressure.

14. The blood pressure measurement system of claim 8, further comprising a wire connectable to the pressure sensing chip for direct electrical connections from a connector of the pressure sensing chip outside a pressure sensing region to a pressure sensing and data processing monitor outside of a pressure sensing region.

15. A method for blood pressure measurement of a patient by attaching a blood pressure measurement device to the patient at a patient measurement site of the patient, the method comprising:
measuring the patient's blood pressure based upon pressure applied by the patient's blood against a pressure transducing member at a first side of the pressure transducing member; and
measuring the patient's blood pressure based upon gravity generated pressures over a height difference between the patient's heart level and a point of blood pressure measurement applied against the pressure transducing member at a second side of the pressure transducing member.

16. The method of claim 15, wherein the pressure transducing member includes a membrane that includes a piezo resistive strain sensor such that a patient's blood abutting against the membrane results in a deformation of the membrane which is measured as a change in resistance in the piezo resistive strain sensor and is measured by a pressure sensing chip for measuring the patient's blood pressure.

17. The method of claim 16, wherein the change in resistance in the piezo resistive strain sensor is measured by the pressure sensing chip with a Wheatstone bridge circuit.

18. The method of claim 15, wherein oil is used as a measuring liquid abutting against the second side of the pressure transducing member to compensate for gravity generated pressures over a height difference between the patient's heart level and the point of blood pressure measurement.

19. The method of claim 15, wherein a liquid with density matched to blood pressure is used as a measuring liquid abutting against the second side of the pressure transducing member to compensate for gravity generated pressures over a height difference between the patient's heart level and the point of blood pressure measurement.

20. The method of claim 15, further comprising a wire connectable to the pressure sensing chip for direct electrical connections from a connector of the pressure sensing chip outside a pressure sensing region to a pressure sensing and data processing monitor outside of a pressure sensing region.
